(19) United States
(12) Patent Application Publication
(10) Pub. No.: US 2021/0030625 A1
(43) Pub. Date: Feb. 4, 2021

(54) DEVICES AND METHODS FOR DETECTING DOSAGE ADMINISTRATION

(71) Applicant: Novartis AG, Basel (CH)

(72) Inventors: Frederick Charles HOUGHTON, West Concord, MA (US); Dhairya Kiritkumar MEHTA, Waltham, MA (US)

(21) Appl. No.: 16/764,794

(22) PCT Filed: Nov. 16, 2018
(86) PCT No.: PCT/US2018/061469

§ 371 Date: May 15, 2020

Related U.S. Application Data
(60) Provisional application No. 62/587,422, filed on Nov. 16, 2017.

(51) Int. Cl. A61J 7/04; A61F 9/00; A61J 1/14

(52) U.S. Cl. CPC A61J 7/0436; A61F 9/0008; A61J 2200/76; A61J 2200/30; A61J 1/1412

(57) ABSTRACT

Systems and methods for detecting administration of a dose of a medicament are described. A medicament dispensing system may include a cap sensor to detect removal of a cap from a container holding a medicament, an orientation sensor to detect an orientation of the container, and a force sensor to detect application of forces to the container. The system may determine that a dose has been administered based on detection of, in order, removal of the cap, movement of the container to an administration orientation, and application of a force to the container greater than a threshold force.

### CROSS-REFERENCE TO RELATED APPLICATIONS

This application claims the benefit of U.S. Provisional Patent Application No. 62/587,422, filed Nov. 16, 2017, the entire contents of which are incorporated herein by reference.

### FIELD

Disclosed embodiments are related to devices and methods for detecting dosage administration of a medicament.

### BACKGROUND

Containers such as bottles are often used to store and dispense one or more doses of a medicament. For example, medicaments such as liquid eye drops may be stored in a bottle that includes an opening such as a nozzle from which the drops may be dispensed.

In some situations, doses of medicament may be administered according to a therapy regimen. The degree of adherence to the therapy regimen may impact the efficacy of the medicament. Accurate monitoring of the administration doses of a medicament from a container may help to determine the degree to which the patient is adhering to the therapy regimen In some cases, healthcare providers may have difficulty determining whether a lack of efficacy of a medication is due to unsuitability of the medication with the particular patient, or due to lack of adherence by the patient to the therapy regimen.

Some conventional medicament containers may lack an ability to monitor the administration of doses of a medicament from a container in an accurate manner. For example, some medicament containers may incorrectly record false positive dose administration events when no actual administration of medication has occurred. The inventors have recognized the need for medicament dispensing systems that accurately monitor the administration of doses of a medicament from a container.

### SUMMARY

In some embodiments, systems and methods for monitoring the administration of doses of a medicament from a container are provided. In some embodiments, systems and methods detect the administration of a dose of a medicament by detecting a sequence of events that corresponds to dispensing the medicament from a container. In some embodiments, systems and methods are arranged to determine an amount of a medicament (e.g., a volume of a liquid medicament) remaining in a container.

According to one embodiment, a method of detecting administration of a dose of medicament from a container is provided. The method comprises, in some embodiments, detecting that a container is in an open configuration, the container holding a medicament and having a dispensing end. The method further comprises detecting that the container is in an administration orientation after detecting that the container is in the open configuration. In the administration orientation, the dispensing end of the container is rotated away from an upwardly facing upright orientation. The method further includes detecting application of a force to a body of the container greater than a threshold force after detecting that the container is in the administration position, and as a result, detecting that administration of a dose of the medicament from the container has occurred.

According to another embodiment, a system for detecting administration of a dose of medicament is provided. The system comprises, in some embodiments, a container holding a volume of medicament, the container having a dispensing end. The system further includes a cap removably coupleable to the container, a cap sensor configured to detect whether the cap is in an open configuration relative to the container, and an orientation sensor configured to detect an orientation of the container. The container has an administration orientation in which the dispensing end of the container is rotated away from an upwardly facing upright orientation. Additionally, the system includes a force sensor configured to detect application of a force to a body of the container and a controller operatively associated with the cap sensor, the orientation sensor, and the force sensor. The controller is configured to detect administration of a dose of the medicament by first detecting, with the cap sensor, that the cap is in the open configuration, then detecting, with the orientation sensor, that the container is in the administration orientation, and then subsequently detecting, with the force sensor, application of a force to the container body, the force greater than a threshold force.

According to yet another embodiment, method of operating a medicament dispensing system is provided. The method comprises, in some embodiments, determining a number of doses of a medicament administered from a container, determining a first volume value of medicament remaining in the container based on the number of doses administered, determining a second volume value of medicament remaining in the container with a volume sensor, and providing a user notification based on a comparison of the first and second volume values.

According to a further embodiment, computer-implemented method of assisting medicament dose administration is provided. The method comprises, in some embodiments, receiving weather forecast data relating to a user's current or future location, and generating on a remote computing device a notification to administer a dose of a medicament based on the weather forecast data.

According to a further embodiment, a computer system comprises a server computer including one or more processors that are configured to receive weather forecast data relating to a user's current or future location, and send the user a notification to administer a dose of a medicament based on the weather forecast data.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a schematic representation of a medicament dispensing system according to one embodiment;

FIG. 2 is a schematic representation of a medicament dispensing system including a cap in a closed configuration on a container, according to one embodiment;

FIG. 3 is a schematic representation of the embodiment of FIG. 2 with the cap in an open configuration;

FIG. 4 is a schematic representation of the embodiment of FIG. 2 with the container moved to an administration orientation;

FIG. 5 is schematic representation of the embodiment of FIG. 2 that illustrates application of force to the container;

FIG. 6 is a perspective view of a medicament dispensing system according to one embodiment;

FIG. 7 is a partially exploded view of the embodiment of FIG. 6;

FIG. 8 is a flow chart depicting a method of detecting administration of a dose of a medicament according to one embodiment;

FIG. 9 is a flow chart depicting a method of using a medicament dispensing system according to one embodiment; and

FIG. 10 is a block diagram of a control circuit according to one embodiment.

### DETAILED DESCRIPTION

The inventors have recognized the need for medicament dispensing systems that monitor the administration of doses of a medicament from a container. The inventors have appreciated that such systems may allow patients and/or caregivers to monitor usage of the medicament, the amount of medicament remaining in a particular container, compliance with a therapy regimen, and/or efficacy of a therapy regimen. Moreover, the inventors have recognized that it may be advantageous for a medicament dispensing system to automatically detect and/or record an administration event such that a user (e.g., a patient) may not have to perform any additional actions to track administration of a dose beyond those actions typically associated with administering a dose from a container. In this manner, a system may track dose administration and may provide information to a patient (e.g., a reminder to take a dose, a notice to order a new container of the medicament, etc.) and/or a caregiver (e.g., information regarding therapy compliance, a notice that a new prescription is needed, etc.) based on the dose administration information detected by the system.

According to some aspects, detecting administration of a dose of a medicament may include detecting a sequence of events that corresponds to dispensing the medicament from a container. For example, dispensing a medicament from a container may involve first removing a cap from the container, then moving the container to an administration orientation, and subsequently applying a force to the container to cause a dose of the medicament to be dispensed from an opening of the container. A medicament dispensing system may include one or more sensors to detect such events associated with dispensing the medicament. The inventors have recognized and appreciated that such systems may permit tracking of dose administration while not requiring contact with the medicament, as described in more detail below.

In one embodiment a medicament dispensing system may include a container (e.g., a bottle containing a liquid medicament such as eye drops) that includes a proximity sensor arranged to detect if a cap has been removed from the container. For instance, once the cap is removed, the container may be in an open configuration to permit dispensing of the medicament from an opening of the container (e.g., a nozzle or other suitable opening). The container may further include an orientation sensor, such as a gyroscope or accelerometer, to detect if the container has been moved into a predetermined administration orientation (e.g., a tilted or an inverted orientation). Moreover, a force or pressure sensor may be provided on the container to detect application of a force (such as a squeezing force) to the container. The various sensors may be coupled to a control circuit that may be configured to detect the various events, and register that a dose has been administered if the events are detected in a predetermined sequence corresponding to administration of a dose. Additionally, in some embodiments, the control circuit may be configured to provide information to a user based on information detected by the sensors. For example, the cap sensor may determine that the cap was not replaced on the container after administering a does, and the control circuit may be configured to send a reminder to the user to replace the cap.

According to some aspects, detecting administration of a dose of a medicament based on detecting an ordered sequence of events may aid in avoiding tracking of false positive dosage events. For example, in some instances, a user may carry a container of a medicament in a bag (e.g., a purse or a backpack), and the container may unintentionally be moved into various orientations (including an administration orientation, such as, in some embodiments, an inverted orientation) and/or squeezed while being carried in the bag. The inventors have appreciated that, in a medicament dispensing system that does not require an ordered sequence of events to register that a dose has been taken, such handling of the container may inadvertently trigger one or more sensors on the container (such as orientation and/or force sensors), which could lead to unintentional registration of dosage administration events. By contrast, requiring detection of a sequence of ordered events to register that a dose has been administered may reduce and/or prevent the possibility that such events could inadvertently be registered as administration of a dose. For example, in some embodiments, a container may not register any input from an orientation and/or force sensor until a cap sensor first detects that a cap has been removed from the container thereby moving the container to an open configuration.

In addition to the above, the inventors have appreciated benefits associated with indirect detection of administration of a dose of a medicament. For example, by detecting a sequence of events corresponding to administration of a dose (e.g., opening a container, moving the container to an administration orientation, and squeezing the container), a medicament dispensing system may be able to detect administration of a dose without directly interacting with and/or contacting the medicament, such as with a flow sensor (e.g., an optical flow sensor) or other type of sensor that must interact with the medicament to detect outflow of the medicament from the container. In some instances, such indirect detection of dose administration while avoiding interaction with the medicament may aid in reducing and/or preventing contaminating, spoiling, or otherwise fouling the medicament. Moreover, in some embodiments, such indirect detection of dose administration may occur as a user administers the dose, such that the user may not have to perform any additional actions to track the administration of the dose other than those actions associated with dispensing the medicament from the container.

According to some aspects, a medicament dispensing system may include a user interface that may permit a patient and/or a caregiver to monitor the administration of doses of the medicament (e.g., to monitor compliance with a therapy regimen, to track an amount of medicament and/or number of doses administered, and/or to track an amount of medicament and/or number of doses remaining in a container). For example, the user interface may include an application provided on a computing device (e.g., a mobile device such as a smartphone or tablet) which may communicate with a control circuit located on a container of the medicament. For instance, when the control circuit detects that a dose has been administered (such as by detecting a specific sequence of events), the container may communicate information regarding the dose administration, such as the time and location of the dose, to the user interface. Alternatively or additionally, a user interface of a medicament dispensing system may provide one or more other functions related to the administration of the medicament, which may include providing a reminder to take a dose of the medicament (e.g., based on time and/or location), indicating that a new container of the medicament may need to be ordered, and/or providing information and/or instructions related to the particular medicament or its administration. In some embodiments, the user interface may include a display unit located on the container. For example, the display unit may be configured to display information such as an expiration date, a number of doses taken, a number of doses remaining, and so on.

In some embodiments, an application (e.g., a mobile phone application, web-based application, or other application used in connection with a suitable computing device) may communicate with one or more other applications or information sources on the computing device to provide one or more functions related to the administration of the medicament. For example, the application may receive calendar and/or weather data available on the computing device and provide indications to the user based on the received data. In some such embodiments, the application may utilize the data to provide a suggestion and/or reminder to take a dose of the medicament based on travel plans and/or current or future weather conditions.

Moreover, in some embodiments, an application may provide one or more additional functions related to the administration of the medicament. For instance, as described in more detail below, the application may provide a symptom tracking functionality, which may allow a user and/or the user's doctor to monitor the efficacy of the medicament as a function of the usage tracked by the medicament dispensing system. Alternatively or additionally, the application may be configured with one or more functions to incentivize or motivate a user to use the medicament according to a therapy regimen. For example, the application may be configured to provide the user with coupons when the user is nearing a need for a refill, and/or the application may allow the user to compare his or her compliance with the therapy regimen to that of the general population, thereby providing a gamification aspect to the user's compliance with the therapy regimen.

According to some aspects, a medicament dispensing system may be arranged to track when a container of a medicament is first opened. For example, it may be desirable to include multiple doses of a medicament within a container but not include any preservatives. The inventors have appreciated that, in some situations, preservatives may be undesirable for one or more reasons. The inventors have appreciated that, in some situations, preservatives may give rise to a negative interaction with the medicament, may cause irritation and/or may have a degree of toxicity. In some such embodiments, the container may be a multi-dose preservative free (MDPF) container, and a user (e.g., a patient and/or caregiver) may dispense multiple doses from the container (e.g., according to a prescribed therapy regimen). However, since preservatives are not included, the medicament may have a limited shelf life after the container is first opened. Accordingly, in some embodiments, cap sensor(s) associated with the container may be used to track when the container is first opened. For example, after the cap is first removed from the container by a user, the system may record the time and date of the opening, and may provide information (such as an expiration date, a date when a new container should be ordered, etc.) based on the date that the container is first opened. Depending on the embodiment, such information may be communicated to the user via a suitable user interface, such as a display on the container and or an application on a smartphone or other suitable computing device.

In addition to the above, in some embodiments, a medicament dispensing system may be arranged to track an amount of time that a container is uncapped or otherwise open. For example, if the container is left open for longer than a predetermined amount of time after administering a dose of the medicament, the system may send a reminder to the user to close the container. Moreover, in some embodiments, the system may be arranged to track the total amount of time that the container is open. For example, a medicament may have a limited shelf life based on the amount of time that the medicament is exposed to an ambient environment. Accordingly, if the total amount of time that the container has remained open exceeds a predetermined amount of time (e.g., corresponding to the shelf life of the medicament), the system may indicate to the user that the medicament has expired and that the container should be discarded and replaced.

According to some aspects, a medicament dispensing system may be arranged to track an amount of a medicament (e.g., a volume of a liquid medicament) remaining in a container. For example, in some embodiments, a system may track the number of doses administered, and may calculate a total volume administered by multiplying the number of doses administered by a volume of a single dose. However, the inventors have appreciated that in some instances, such a volume measurement may not provide a desired degree of accuracy, for example, due to variations in the amount of medicament dispensed in a single dose. Accordingly, in some embodiments, a medicament dispensing system may include one or more volume sensors arranged to measure the volume of medicament remaining in the container. For example, the volume sensor(s) may include fluid level sensors such as capacitive or other suitable non-contact sensors arranged to measure the volume of medicament remaining in the container without contacting the medicament. In some embodiments, a volume sensor may be arranged to cooperate with an orientation sensor such that the volume is only measured when the container is in an upright orientation.

Moreover, the inventors have appreciated that it may be desirable for a medicament dispensing system to determine a volume remaining in a container in more than one manner. For example, measuring the volume with a volume sensor as discussed above may require the container to be in a specific orientation, such as an upright orientation. Accordingly, the inventors have appreciated that it may be advantageous to determine the volume in a second manner that does not require the container to be in a specific orientation, such as based on a number of doses administered from the container.

In addition to the above, the inventors have appreciated that determining a volume remaining in a container in more than one manner may allow a medicament dispensing system to provide information to a user based on volume values measured in different ways. For example, a system may determine a first volume value based on the number of doses administered (as discussed above,) and a second volume value based on a measurement from one or more volume sensors. The system may compare the volume measurements and provide information to a user based on the comparison. For example, if the second volume value is larger than the first volume value, the system may determine that the user is not dispensing enough of the medicament with each dose administration (e.g., due to the container not being in the correct administration orientation and/or inadequate pressure being applied to the container while administering a dose). Conversely, if the second volume value is smaller than the first volume value, the system may determine that the user is dispensing too much medicament with each dose administration (e.g., by applying too much pressure to the container). Depending on the particular embodiment, the system may indicate to a user (e.g., via a suitable user interface) if a discrepancy in the first and second volume values is detected, and in some instances, the system may provide additional instructions to the user to aid in correcting any user error associated with dispensing the medicament.

According to some embodiments, a medicament dispensing system may further include a temperature sensor configured to measure the temperature of the medicament contained within the medicament dispensing system, and the system may provide information to the user based on the measured temperature. For example, the system may detect that the temperature of the medicament is above or below an optimal storage temperature or temperature range, and the system may provide a reminder to the user to store the medicament at the proper temperature.

Turning now to the figures, specific non-limiting embodiments of medicament dispensing systems and associated methods are described in further detail. It should be understood that the various systems, components, features, and methods described relative to these embodiments may be used either individually and/or in any desired combination as the disclosure is not limited to only the specific embodiments described herein. Moreover, for the sake of clarity, the disclosed embodiments are described in connection with containers to dispense liquid medicaments such as eye drops. However, it should be understood that the systems and methods described herein may be employed with other forms of medicaments, as the current disclosure is not limited in this regard.

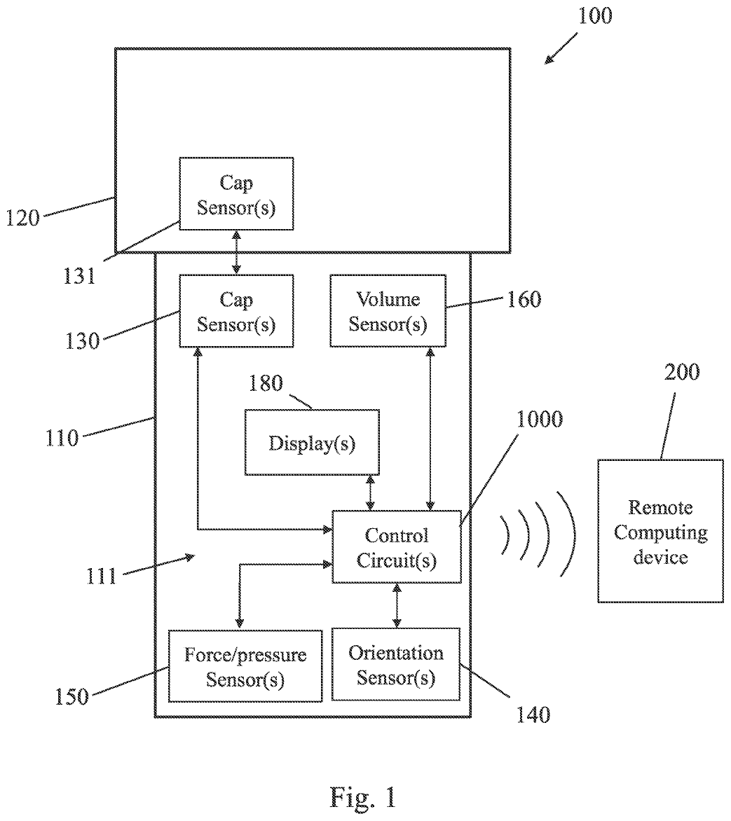

FIG. 1 is a schematic representation of a medicament dispensing system 100. The dispensing system includes a container 110, which may include an internal cavity 111 in which a volume of a medicament such as liquid eye drops may be contained. A cap 120 is removably received on the container. For example, the cap 120 may be selectively moved from a closed configuration in which the cap is received on the container 110 and the container is in a closed configuration, and an open configuration in which the cap is removed from the container such that the container is in open configuration. As described in more detail below, removing the cap may expose an opening on the container such as a nozzle or other suitable dispensing structure from which the medicament held within the container (e.g., within internal cavity 111) may be dispensed. It should be understood that the cap 120 may be attached and/or secured to the container in any suitable manner, including, but not limited to, a threaded interface, a snap-fit interface, and or a friction-fit interface. In some embodiments, the cap may be connected to the container via a hinge (such as a living hinge), tether or other suitable structure that may aid in avoiding misplacement of the cap when the cap is removed from the container. Moreover, while a container with a separate cap that is removable from a container is depicted in the figures, it should be understood that other structures for moving the container between a closed configuration and an open configuration may be suitable, including, but not limited to, a valve that cooperates with an opening of the container to selectively open and close the container. Accordingly, as used herein, a cap may include any suitable structure movable relative to a container to move the container between the open configuration and the closed configuration.

The medicament dispensing system 100 includes a plurality of sensors arranged to detect various aspects associated with dispensing the medicament from the container 110. For example, one or more cap sensors 130 and 131 may be arranged to detect if the cap 120 is removed from the container 110. In the depicted embodiment, the cap sensors include a first cap sensor 130 located on the container 110 and a second cap sensor 131 located on the cap 120, and the first and second cap sensors may cooperate to determine if the cap has been removed from the container. In one embodiment, the cap sensors may be arranged as a proximity sensor to detect if the cap 120 is proximate to the container. For example, the cap sensors 130 and 131 may include cooperating magnetic, capacitive, inductive, optical (e.g., infrared) elements, and/or any other suitable type of proximity sensor, and the sensors may be operatively coupled to a control circuit 1000 (discussed in more detail below) and may send a signal to the control circuit when the sensors detect that the cap is removed from the container.

The medicament dispensing system 100 may include one or more orientation sensors 140 arranged to sense the orientation of the container 110, for example, to detect if the container is in an administration orientation. Depending on the embodiment, the orientation sensor(s) may include accelerometers, gyroscopes, inertial measurement units, and/or any other suitable sensors to detect the orientation of the container 110.

In some embodiments, the medicament dispensing system 100 may include one or more force or pressure sensors 150 that may be arranged to detect application of force or pressure to the container 110, such as due to a user squeezing the container to dispense a dose of medicament therefrom. Suitable force/pressure sensors include, but are not limited to, resistive force sensors (e.g., force-sensitive resistive elements), capacitive force sensors, strain gauges, and/or piezoelectric elements. In some embodiments, the force/pressure sensor(s) 150 may extend partially or completely around the exterior of the container such that a force applied to any suitable portion of the container during administration of a dose may be detected by the force/pressure sensor(s).

Similar to the cap sensors 130 and 131, the orientation sensor(s) 140 and force/pressure sensor(s) 150 may be operatively coupled to the control circuit 1000 and may send signals to the control circuit when the sensors detect one or more events associated with dispensing a dose of the medicament. For example, the orientation sensor(s) 140 may send a signal to the control circuit when the orientation sensor detects that the container 110 has been moved to a dispensing orientation, such as a partially or fully inverted orientation in which a dispensing portion of the container is oriented downwardly. Similarly, the force/pressure sensor(s) 150 may send a signal to the control circuit upon when a force applied to the container 110 exceeds a threshold force, such as a force required to dispense a dose of medicament from the container.

In some embodiments, the medicament system 100 may include a volume sensor 160 arranged to measure a volume of medicament remaining in the container 110. As discussed previously, in some embodiments, such a volume measurement may be used to determine a second volume value that may be compared to a first volume value calculated from the number of doses administered and a volume of each dose. The first and second volume values may be compared to determine if the medicament is being dispensed properly (e.g., if the proper amount of medicament is being dispensed with each dose). In some embodiments, the volume sensor(s) 160 may include a capacitive fluid level sensor located on the exterior of the container 110, which may be arranged to measure the volume of the medicament without contacting the medicament. In other embodiments, the volume sensor(s) 160 may include inductive volume sensors, or any other suitable sensors to measure the volume of medicament remaining in the container. Moreover, in certain embodiments, the volume sensor(s) 160 may cooperate with the orientation sensor(s) 140 and/or control circuit 1000 such that the volume sensor(s) are only activated to measure the volume when the container 110 is in a predetermined orientation, such as an upright configuration. For example, measuring the volume only when the container is in an upright orientation may aid in providing accurate volume measurements.

Moreover, in some embodiments, a display 180 such as a flexible display unit may be provided on the container, and may be arranged to display information regarding the medicament, such as the number of doses remaining, an expiration date for the medicament, and/or reminder(s) or other suitable notifications.

As noted previously, the medicament delivery system 100 may include a control circuit 1000 that is operatively coupled to the various sensors, and the sensors may communicate signals to the control circuit based on detection events associated with administration of a dose of the medicament, and/or to control one or more additional functions of the system such as measuring the remaining volume of medicament with a volume sensor 160. As illustrated in FIG. 1, the control circuit may include a communication system to permit communication with a remote computing device 200, such as a smartphone, tablet and/or other suitable communication gateway. In some embodiments, such communication may be wireless, for example, via a suitable wireless communication protocol such as Bluetooth, Wi-Fi, or a cellular network. Depending on the embodiment, such communication between the control circuit 1000 and the remote computing device 200 may allow one or more aspects of the user interface of the medicament dispensing system to be implemented on the remote computing device 200. For example, the remote computing device may include software such as a mobile application configured to communicate with the system 100, and the application may be configured to track dose administration and/or compliance with a therapy regimen, and/or to provide information to a user such as a reminder to take a dose or an indication that the medication has expired or will be expiring soon.

In some embodiments, the medicament dispensing system 100 may be arranged to utilize one or more functions of the remote computing device 200 and/or mobile app, such as a location tracking functionality, in combination with the functions discussed above that may be performed by the system 100. For example, in one embodiment, the system 100 may utilize location tracking functionality (e.g., GPS tracking) of the remote computing device track the location where doses are administered after determining the location of a user with the remote computing device. However, it should be understood that the current disclosure is not limited to tracking the location where doses are administered only via the remote computing device 200. For example, in some embodiments, the control circuit 1000 may include GPS or other suitable location tracking sensors to track where doses are administered. Moreover, in certain embodiments, location tracking functionality on the remote computing device 200 may cooperate with location tracking functionality of the control circuit 1000. For example, such a system may notify a user (e.g., on the remote computing device 200) if the user leaves a location without the medicament dispensing system 100.

In addition to the above, in some embodiments, location tracking functionality of a medicament dispensing system (e.g., on a remote computing device and/or on a control circuit) may be utilized to provide contextual information to a user based on the user's location. For example, such information may include changes to a dosage regimen or other suitable user instructions (e.g., administration instructions) based on weather, elevation, or other environmental conditions at a particular location.

In further embodiments, an application or other suitable interface on the remote computing device 200 may communicate with other applications and/or data sources on the remote computing device to provide one or more additional functions. For example, the remote computing device 200 may contain a user's calendar data (e.g., in a calendar application), which may include information related to where a user plans to travel. Based on this data, and in some embodiments, based also on data from the system 100, the application may provide treatment suggestions and/or treatment reminders to the user. Similarly, in some embodiments, the remote computing device 200 may include weather data, such as current weather conditions or weather forecasts, and the application may provide the user treatment suggestions based on the weather data. In one exemplary embodiment in which the medicament dispensing system is configured to dispense eye drops for dry-eye related conditions, such calendar and weather based recommendations may advantageously allow the systems described herein to provide suggestions based on the climate at a current or future location of the user. In particular, the amount of medicament needed for a particular user may depend on weather conditions such as the humidity level, and thus the systems described herein may utilize the weather and/or calendar information to proactively recommend an appropriate therapy regimen to the user.

As discussed previously, an application or other suitable interface on the remote computing device may be further configured to provide one or more additional functions. For example, the application may include a symptom journal function that may allow a user to track symptoms, and the system may associate the reported symptoms with data related to dose administration. In some instances, such arrangements may allow the system to evaluate the efficacy of the medicament, and information regarding the efficacy may be communicated to the user's doctor (e.g., to adjust a treatment protocol, if needed). In some embodiments, such a symptom journal function may present the user with a questionnaire with specific questions related to the user's treatment. Alternatively or additionally, the application may provide one or more functions related to the use of a particular medicament in connection with the dispensing systems described herein. For example, in connection with a medicament to treat dry eye or similar eye-related conditions, the application may provide reminders for a user to perform eye exercises at specified time intervals.

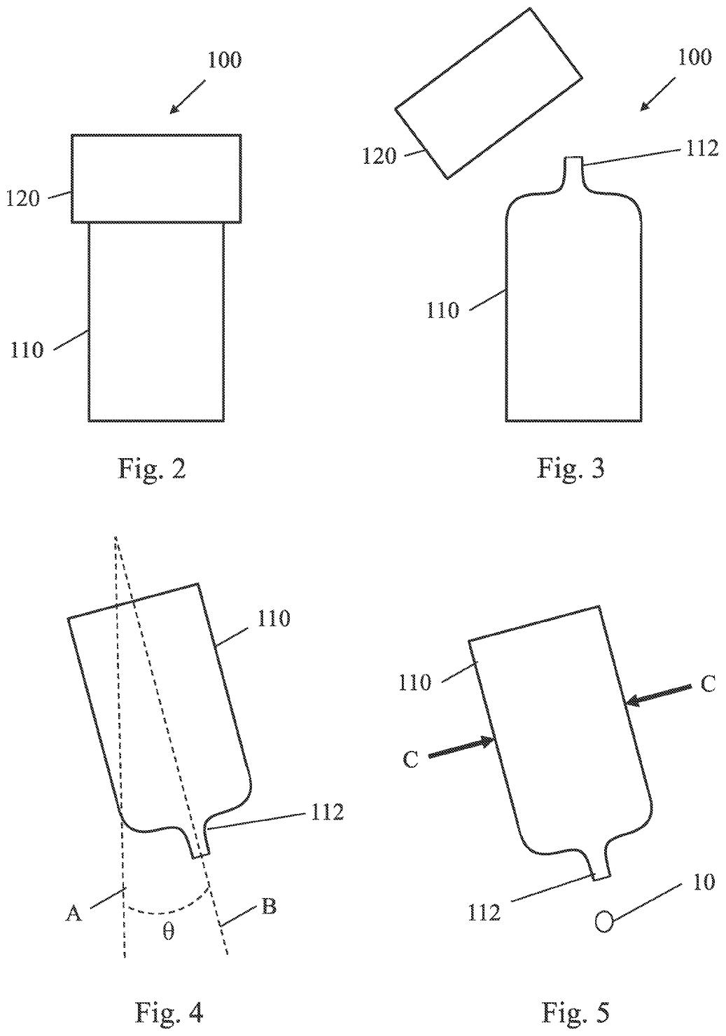

Referring now to FIGS. 2-5, an exemplary method of detecting administration of a dose of medicament from the system 100 is described in more detail. In particular, FIG. 2 depicts the system 100 in a configuration prior to administration of a dose, with the cap 120 received on the container 110 such that the container is in a closed configuration. As illustrated in FIG. 3, a first step of administering a dose includes opening the container, such as by removing the cap 120 from the container. Opening the container may include moving the cap 120 from a closed configuration to an open configuration, which may be detected by the cap sensors 130 and 131, and as discussed previously, the cap sensors may send a signal to the control circuit 1000 to indicate that the container has been opened. Moreover, opening the container (e.g., by removing the cap 120) may expose a dispensing portion such as a nozzle 112 on the container from which the medicament may be dispensed.

As depicted in FIG. 4, after the cap 120 is removed and the container is in an open configuration, the container 110 may be moved to an administration orientation in which the nozzle 112 is oriented downwardly such that a longitudinal axis B of the container 110 is oriented at an angle θ relative to a vertical axis A. Once in the administration orientation, the orientation sensor(s) 140 may send a corresponding signal to the controller, as discussed previously. However, it should be understood that the current disclosure is not limited to any particular angle or range of angles for the administration orientation. For instance, the administration orientation may correspond to a completely inverted orientation (i.e., a 0 value of 0°), or a range of angles, such as −30° to 30°, −45° to 45°, −60° to 60°, or −90° to 90° relative to the vertical axis A. Additionally, in some embodiments, the system 100 may provide a notification to the user to indicate that the medicament is ready to be dispensed once the orientation sensor(s) 140 detect that the container 110 has been moved to the administration orientation. For example, the indication may include an audible, visual, and/or tactile indication.

As illustrated in FIG. 5, after the cap 120 is removed to change the container 110 to the open configuration and the container is in the administration orientation, a user may apply a force and/or pressure to the container to cause one or more drops 10 of medicament to be dispensed from the container. For example, the force and/or pressure may be applied by squeezing the container along directions C. As discussed previously, if the force/pressure is greater than a predetermined threshold pressure, the force/pressure sensor(s) 150 may send a signal to the control circuit 1000. Once the controller receives the respective signals from the cap sensors 130 and 131, orientation sensor(s) 140, and force/pressure sensor(s) 150 in that order, the controller may record that a dose has been administered.

In some embodiments, the control circuit may selectively operate and/or provide power to one or more sensors depending on signals from other sensors. For example, a medicament dispensing system 100 may include a battery to power the various components, and such selective operation of the sensors may aid in prolonging the battery life and/or avoiding recording false positive dose administration events. In one embodiment, the control circuit may maintain the orientation sensor(s) 140 and force/pressure sensor(s) 150 in a powered-off state or a low-power standby state until the cap sensor(s) 130 and 131 determine that the cap has been removed from the container. Subsequently, the orientation sensor(s) 140 may be powered on, but the force/pressure sensor(s) 150 may remain powered off or in a low-power standby state. Once the orientation sensor(s) determine that the container is in an administration orientation, the control circuit may power on the force/pressure sensors to determine if a force/pressure applied to the container exceeds a threshold force/pressure corresponding to administration of a dose. Moreover, in embodiments including a volume sensor 160, the volume sensor may remain powered off or in a lower-power standby state unless the orientation sensor determines that the container is in an upright orientation and a volume measurement is needed. In some such embodiments, the control circuit may intermittently operate the orientation sensor to determine if the container is in the upright position, for example, according to a predetermined measurement schedule or after a predetermined amount of time after an earlier measurement and/or dose administration.

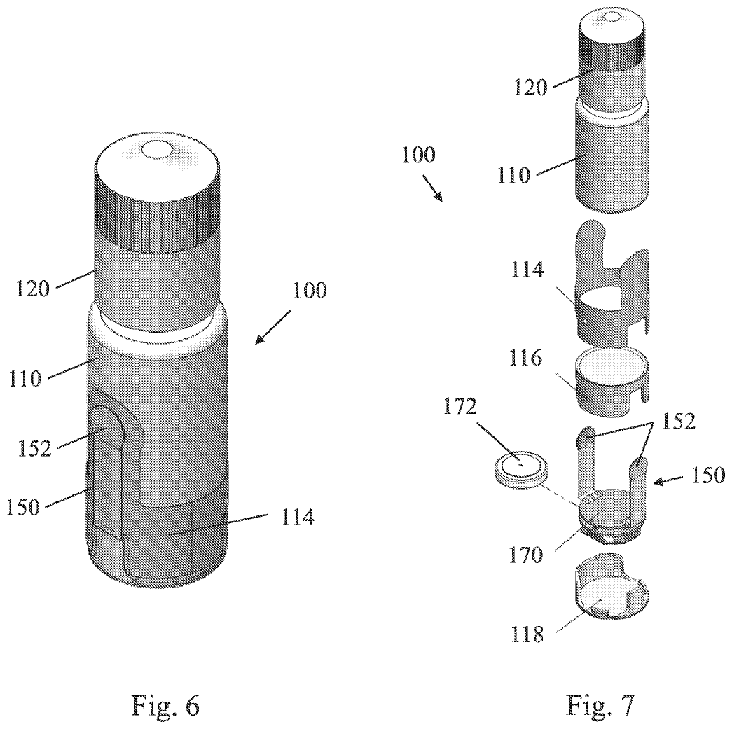

FIGS. 6-7 depict a perspective view and a partially exploded view, respectively, of another embodiment of a medicament dispensing system 100. Similar to the embodiments described above, the system 100 includes a container 110 and a cap 120 removably received on the container 110. In the depicted embodiment, a pressure sensor 150 includes a pair of pressure sensitive pads 152 positioned on opposing sides of the container 110, however, embodiments in which one or more pressure sensors are provided around the entire circumference of the container 110 are also contemplated. Moreover, a printed circuit board assembly (PCBA) 170 is provided between a base 116 and an end cap 118. The base and cap may be attached to the container 110 via an adhesive sticker 114 or by other suitable connection arrangements such as a threaded or snap-fit interface. The PCBA 170 may include an orientation sensor and control circuit, such as those discussed above in connection with FIGS. 1-5. Additionally, a battery 172 may be connected to the PCBA 170 to provide power to the system 100, though it should be understood that other power sources, such as kinetic energy recovery systems, wireless power systems, or any other suitable power system, may be used to provide power to the various components of the system, as the disclosure is not limited in this regard.

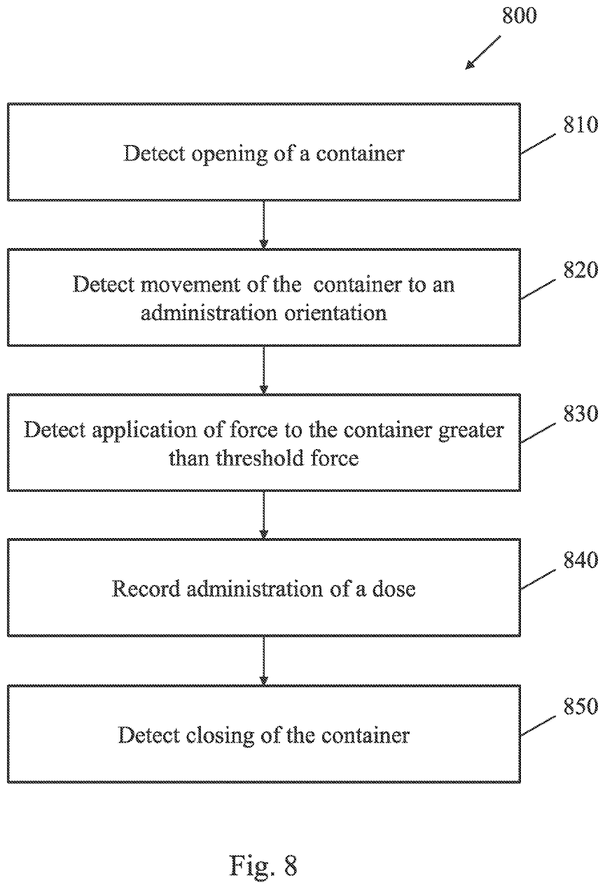

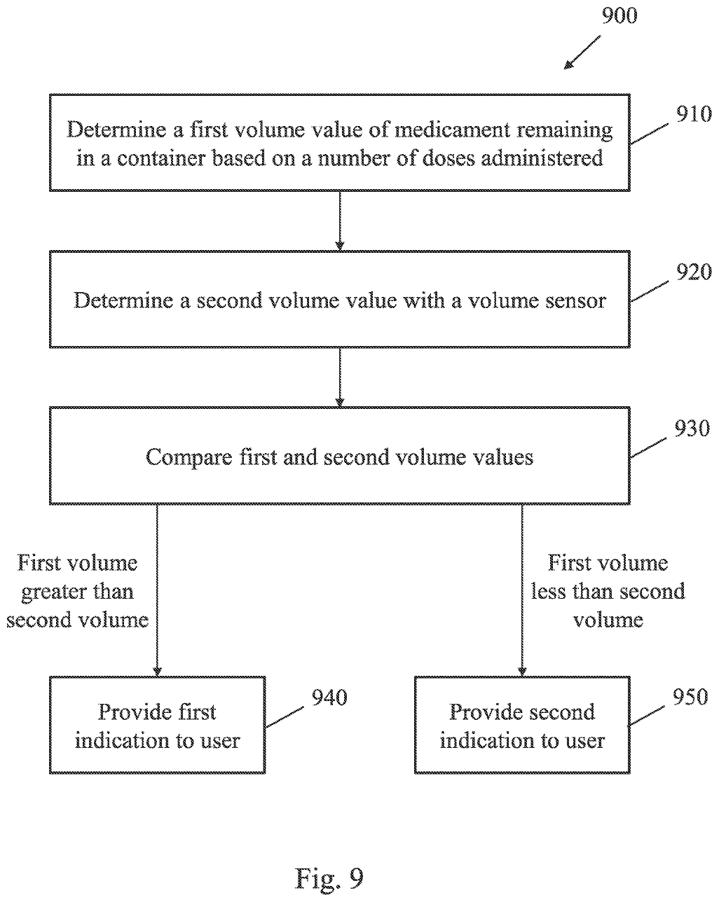

Having described various aspects of medicament dispensing systems, exemplary embodiments of their methods of use are described in more detail in connection with FIGS. 8-9.

FIG. 8 is a flow chart of an one embodiment of a method 800 of detecting administration of a dose of medicament from a container based on an ordered sequence of events detected by sensors on a container. At step 810, opening of a container is detected, for example, with one or more cap sensors, such as proximity sensors. After the opening of the container is detected, movement of the container to an administration orientation (e.g., an inverted orientation) is detected at step 820 using one or more orientation sensors, such as accelerometers and/or gyroscopes. Subsequently, at step 830, a force sensor is used to detect if a force applied to the container (e.g., due to a user squeezing the container) exceeds a threshold force. Once each of these events is detected in order, administration of a dose of the medicament is recorded at step 840. In some embodiments, the method may further include detecting that the container is moved back to the closed configuration at step 850 after a dose has been administered, for example, by detecting that the cap has been replaced on the container.

FIG. 9 is a flow chart of an exemplary embodiment of a method 900 of operating a medicament dispensing system. At step 910, a first volume value of medicament remaining in a container is determined based on a number of doses administered from the container. For example, the number of doses may be determined based on detecting an ordered sequence of events corresponding to administration of the medicament, and the first volume value may be calculated by multiplying the determined number of doses by a nominal dose volume, and subtracting the volume of medicament administered from an initial volume provided in the container. At step 920, a second volume value is determined by measuring the volume of the medicament in the container with a suitable volume sensor, such as a capacitive fluid level sensor, as discussed above. At step 930, the first and second volume values are compared. If the first and second volume values differ by more than a predetermined threshold difference (e.g., by more than a permissible margin of error), the system may provide a suitable indication to the user. In particular, if the first volume value is greater than the second volume value, a first indication may be provided to a user at step 940. For example, the first indication may be a notice that the user is not applying enough force to the container during administration of the drug such that the actual volume dispensed with each dose is less than the nominal dose volume that is intended to be dispensed with each dose administration. Correspondingly, the second volume value (measured by the volume sensor) may be greater than a first volume value based on the number of doses administered. Similarly, if the first volume value is less than the second volume value, a second indication may be provided to the user at step 950. For example, the second indication may include a notice that the user is applying too large of a force to the container during administration of doses, such that the actual volume dispensed is greater than the nominal dose volume intended to be dispense. In some embodiments, a medicament dispensing system may provide further instructions to the user along with the first or second indications, such as instructions regarding the proper administration of the medicament.

In some embodiments, methods described herein may be carried out using one or more control circuits, such as control circuit 1000 depicted in FIG. 1. However, it should be understood that the embodiments described herein are not limited to operating with any particular type of control circuit.

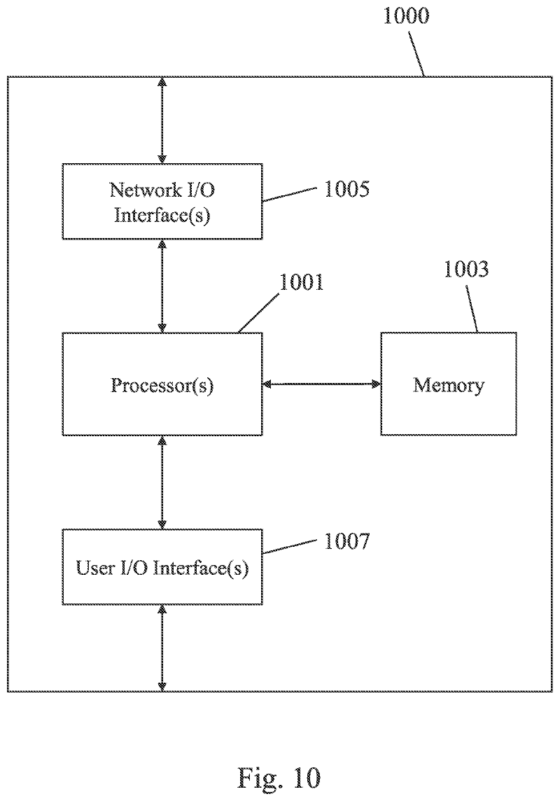

FIG. 10 is a block diagram of an illustrative control circuit 1000 that may be used to implement any of the above-described methods. Control circuit 1000 may include one or more processors 1001 and one or more tangible, non-transitory computer-readable storage media (e.g., memory 1003). Memory 1003 may store, in a tangible non-transitory computer-recordable medium, computer program instructions that, when executed, implement any of the above-described functionality. Processor(s) 1001 may be coupled to memory 1003 and may execute such computer program instructions to cause the functionality to be realized and performed.

Control circuit 1000 may also include a network input/output (I/O) interface 1005 via which the computing device may communicate with other computing devices (e.g., over a network), and may also include one or more user I/O interfaces 1007, via which the computing device may provide output to and receive input from a user. The user I/O interfaces may include devices such as a display device (e.g., a monitor or touch screen) located on a medicament container and/or on a separate computing device such as a smartphone or tablet, speakers, a microphone, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more control circuits can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A method of detecting administration of a dose of medicament from a container, the method comprising:
detecting that a container is in an open configuration, the container holding a medicament and having a dispensing end;
detecting that the container is in an administration orientation after detecting that the container is in the open configuration, wherein in the administration orientation, the dispensing end of the container is rotated away from an upwardly facing upright orientation; and
detecting application of a force to a body of the container greater than a threshold force after detecting that the container is in the administration orientation, and as a result, detecting that administration of a dose of the medicament from the container has occurred.

2. The method of claim 1, further comprising detecting closure of the container from the open configuration to a closed configuration after detecting application of the force to the body of the container.

3. The method of claim 2, further comprising generating a notification if the container remains in the open configuration over a predetermined period of time.

4. The method of claim 1, wherein detecting that the container is in an open position includes detecting that a cap is in an open position relative to the container.

5. (canceled)

6. The method of claim 4, further comprising detecting movement of the cap relative to the container from the open configuration to a closed configuration.

7. (canceled)

8. (canceled)

9. (canceled)

10. (canceled)

11. (canceled)

12. The method of claim 1, further comprising detecting a volume of medicament remaining in the container.

13. The method of claim 12, further comprising determining that the container is in an upright position prior to measuring volume.

14. The method of claim 1, further comprising generating a notification to indicate that the medicament is ready to be dispensed after detecting that the container is in the administration orientation.

15. (canceled)

16. The method of claim 1, further comprising detecting a first time the container is in the open configuration and generating a notification based on an amount of time elapsed since the container was first in the open configuration.

17. A system for detecting administration of a dose of medicament, the system comprising:
a container holding a volume of medicament, the container having a dispensing end;
a cap removably coupleable to the container
a cap sensor configured to detect whether the cap is in an open configuration relative to the container;
an orientation sensor configured to detect an orientation of the container, the container having an administration orientation in which the dispensing end of the container is rotated away from an upwardly facing upright orientation;
a force sensor configured to detect application of a force to a body of the container; and
a controller operatively associated with the cap sensor, the orientation sensor, and the force sensor, wherein the controller is configured to detect administration of a dose of the medicament by first detecting, with the cap sensor, that the cap is in the open configuration, then detecting, with the orientation sensor, that the container is in the administration orientation, and then subsequently detecting, with the force sensor, application of a force to the body of the container, the force greater than a threshold force.

18. The system of claim 17, wherein the controller is configured to detect movement of the cap from the open configuration to a closed configuration after detecting administration of the dose of the medicament.

19. (canceled)

20. The system of claim 17, wherein the cap sensor includes a proximity sensor.

21. (canceled)

22. (canceled)

23. (canceled)

24. The system of claim 17, further comprising a volume sensor located on the body of the container and configured to measure a volume of medicament remaining in the container when the container is in the upwardly facing upright orientation.

25. The system of claim 24, wherein the volume sensor includes a capacitive fluid level sensor.

26. (canceled)

27. The system of claim 26, wherein the controller is configured to detect at least one of a total amount of time the cap is in the open configuration and a number of times the cap has been moved to the open configuration.

28. (canceled)

29. (canceled)

30. The system of claim 17, wherein the controller is configured to detect a first time the cap is in the open configuration and to generate a notification based on an amount of time elapsed since the cap was first in the open configuration.

31. A method of operating a medicament dispensing system, the method comprising:
determining a number of doses of a medicament administered from a container;
determining a first volume value of medicament remaining in the container based on the number of doses administered;
determining a second volume value of medicament remaining in the container with a volume sensor; and
providing a user notification based on a comparison of the first and second volume values.

32. (canceled)

33. (canceled)

34. (canceled)

35. (canceled)

36. (canceled)

37. (canceled)

38. (canceled)

39. A computer-implemented method of assisting medicament dose administration, the method comprising:
receiving weather forecast data relating to a user's current or future location; and
generating on a remote computing device a notification to administer a dose of a medicament based on the weather forecast data.

40. The method of claim 39, wherein the medicament is selected for treatment of dry eye.

41. (canceled)

42. (canceled)

43. (canceled)

44. (canceled)

45. A computer system, comprising:
a server computer including one or more processors that are configured to:
receive weather forecast data relating to a user's current or future location; and
send the user a notification to administer a dose of a medicament based on the weather forecast data.

46. The computer system of claim 45, wherein the medicament is selected for treatment of dry eye.

47. (canceled)

48. (canceled)

49. (canceled)

50. (canceled)
